(19) United States
(12) Patent Application Publication
(10) Pub. No.: US 2019/0269943 A1
(43) Pub. Date: Sep. 5, 2019

(54) LIMITED USE ULTRASONIC COUPLING DEVICE

(71) Applicant: ZetrOZ Systems LLC, Trumbull, CT (US)

(72) Inventors: George K. LEWIS, JR., Trumbull, CT (US); Shane FLESHMAN, Milford, CT (US); Matthew D. LANGER, Milford, CT (US)

(73) Assignee: ZetrOZ Systems LLC, Trumbull, CT (US)

(21) Appl. No.: 16/337,379

(22) PCT Filed: Feb. 25, 2015
(86) PCT No.: PCT/US2015/017596

§ 371 Date: Mar. 27, 2019

Related U.S. Application Data
(60) Provisional application No. 61/944,525, filed on Feb. 25, 2014.

(51) Int. Cl. A61N 7/00; A61B 8/00; G10K 11/02

(52) U.S. Cl. CPC A61N 7/00; A61B 2090/0814; G10K 11/02; A61B 8/4281

(57) ABSTRACT

The present invention relates to ultrasound coupling adapters, ultrasound coupling devices, ultrasound coupling systems, and methods of using the adapters, coupling devices, and coupling systems in various ultrasound applications. In one embodiment, the ultrasound coupling adapter is for coupling an ultrasound transducer to an ultrasound coupling medium. The ultrasound coupling adapter comprises an interface support region for operably interfacing the ultrasound transducer to the ultrasound coupling medium; and an integrated means for (i) rendering the ultrasound coupling adapter inoperable, and/or (ii) preventing operation of the ultrasound transducer by keeping it from being properly coupled with the ultrasound coupling adapter and/or ultrasound coupling medium.

### CROSS-REFERENCE TO RELATED APPLICATIONS

This application claims priority benefit of U.S. Provisional Patent Application Ser. No. 61/944,525, filed Feb. 25, 2014, the disclosure of which is hereby incorporated by reference herein in its entirety.

### FIELD OF THE INVENTION

This disclosure relates to, inter alia, an ultrasonic coupling device. More particularly, the present invention relates to a limited use ultrasonic coupling device as disclosed or contemplated herein. The present invention further relates to methods of using the limited use ultrasonic coupling device of the present invention.

### BACKGROUND OF THE INVENTION

Ultrasound has been used for over 60 years and is one of the most widely and frequently used electrophysical agents. Acoustic waves mechanically stimulate tissues, triggering biological effects including the down regulation of inflammatory cytokines, increased transport kinetics, protein synthesis, and extracellular matrix deposition. It has been used to treat pain, musculoskeletal injuries, and to promote soft-tissue and wound healing.

Until now, ultrasound has been primarily confined to the clinician's office, where a trained professional applies ultrasound and monitors the diagnostic, imaging, or therapeutic regimen. Therefore, devices can exist with limited safety precautions and very little to no functionality built-in for an average patient. U.S. Patent Application Publication No. 2012/0283605 discloses a portable ultrasound system that is designed to be wearable for long duration treatments. U.S. Patent Application Publication No. 2012/0277640 and U.S. Patent Application Publication No. 2013/0144193 are directed towards wearable coupling methods utilizing ultrasound coupling media. To date, other such coupling methods disclosed do not include any safety precautions against customer misuse. Furthermore, many coupling methods are not designed for adaptability to different body types and locations on the body.

The present invention incorporates mechanical, electromechanical, chemical, optical, and/or electrical means to limit the use of an ultrasound system. With self-applied, unmonitored, long duration, ultrasound coupling devices, the ultrasound coupling media will lose moisture, shrivel, or become less efficient at transmitting ultrasound as time progresses. Therefore, a means of preventing further use of inefficient coupling devices is required. This serves a two-fold purpose: coupling media that becomes less transmissive to ultrasound will (i) prevent effective ultrasound treatment and (ii) increase risk of injury to the patient by increasing thermal dissipation and thus the temperature of the coupling device.

The present invention is directed to overcoming these and other deficiencies in the art.

### SUMMARY OF THE INVENTION

The present invention relates to ultrasound coupling adapters, ultrasound coupling devices, ultrasound coupling systems, and methods of using the adapters, coupling devices, and ultrasound coupling systems in various ultrasound applications.

In one aspect, the present invention provides an ultrasound coupling adapter for coupling an ultrasound transducer to an ultrasound coupling medium. The ultrasound coupling adapter comprises: an interface support region for operably interfacing the ultrasound transducer to the ultrasound coupling medium; and an integrated means for (i) rendering the ultrasound coupling adapter inoperable, and/or (ii) preventing operation of the ultrasound transducer when not properly coupled with the ultrasound coupling adapter and/or ultrasound coupling medium.

In another aspect, the present invention provides an ultrasound coupling device that comprises: an ultrasound coupling adapter according to the present disclosure; and an ultrasound coupling medium housed in the ultrasound coupling adapter. The integrated means is for rendering the ultrasound coupling adapter inoperable, thereby rendering the ultrasound coupling device inoperable. In certain embodiments, the ultrasound coupling device further comprises an adhesive fabric for interfacing the ultrasound coupling device with a subject, where the fabric has adhesive properties that substantially diminish after first use by the subject. In certain other embodiments, the ultrasound coupling device further comprises an adhesive fabric for interfacing the ultrasound coupling device with a subject, where the fabric has adhesive properties that diminish by 50% or greater using the ASTM D903 standard.

In another aspect, the present invention provides an ultrasound coupling system comprising: an ultrasound coupling device according to the present disclosure; and an ultrasound transducer configured for operable attachment to the ultrasound coupling device.

In another aspect, the present invention provides a method of regulating application of ultrasound energy to a subject, where the method comprises the steps of: applying ultrasound energy to a subject using an ultrasound coupling system of the present disclosure; and manipulating the integrated means of the ultrasound coupling adapter so as to render the ultrasound coupling device inoperable, thereby causing the ultrasound energy to cease being applied to the subject.

In another aspect, the present invention provides an ultrasound coupling device comprising: an ultrasound coupling adapter according to the present disclosure; and an ultrasound coupling medium housed in the ultrasound coupling adapter, where the integrated means is for preventing operation of an ultrasound transducer when not properly coupled with the ultrasound coupling adapter and/or ultrasound coupling medium.

In another aspect, the present invention provides an ultrasound coupling system comprising: an ultrasound coupling device according to the present disclosure; and an ultrasound transducer configured for operable attachment to the ultrasound coupling device.

In another aspect, the present invention provides a method of regulating application of ultrasound energy to a subject, where the method comprises the steps of: (i) operably coupling the ultrasound transducer to the ultrasound coupling adapter and/or ultrasound coupling medium; (ii) such that said operably coupling manipulates the integrated means; (iii) whereby the manipulation of the integrated means allows activation of the ultrasound coupling system; and (iv) applying ultrasound energy to a subject using the activated ultrasound coupling system.

In another aspect, the present invention provides an ultrasound coupling device comprising: an ultrasound coupling adapter according to the present disclosure; and an ultrasound coupling medium housed in the ultrasound coupling adapter, where the integrated means is for both (i) rendering the ultrasound coupling adapter inoperable and (ii) preventing operation of the ultrasound transducer when not properly coupled with the ultrasound coupling adapter and/or ultrasound coupling medium.

In another aspect, the present invention provides an ultrasound coupling system comprising: an ultrasound coupling device according to the aspect of the present disclosure as described in the preceding paragraph; and an ultrasound transducer configured for operable attachment to the ultrasound coupling device.

In another aspect, the present invention provides a method of regulating application of ultrasound energy to a subject, the method comprising the steps of: applying ultrasound energy to a subject using an ultrasound coupling system according to the aspect of the present disclosure as described in the preceding paragraph; and manipulating the integrated means of the interface support region of the ultrasound coupling device so as to (i) render the ultrasound coupling device inoperable and (ii) prevent operation of an ultrasound transducer when not properly coupled with the ultrasound coupling adapter and/or ultrasound coupling medium.

In another aspect, the present invention provides an ultrasound coupling device comprising: an ultrasound coupling adapter according to the present disclosure; and an ultrasound coupling medium housed in the ultrasound coupling adapter, where the ultrasound coupling device in a first state is operably connected to an ultrasound transducer and comprises an integrated means of rendering the ultrasound coupling device inoperable, and where the ultrasound coupling device in a second state is inoperable.

In another aspect, the present invention provides a method of regulating application of ultrasound energy to a subject, the method comprising the steps of: applying ultrasound energy to a subject using an ultrasound coupling system comprising an ultrasound transducer coupled to the ultrasound coupling device according to the aspect of the present disclosure as described in the preceding paragraph, where ultrasound energy is applied when the ultrasound coupling device is in the first state, and where ultrasound energy is not applied when the ultrasound coupling device is in the second state.

These and other objects, features, and advantages of this invention will become apparent from the following detailed description of the various aspects of the invention taken in conjunction with the accompanying drawings.

### BRIEF DESCRIPTION OF THE DRAWINGS

For the purpose of illustrating aspects of the present invention, there are depicted in the drawings certain embodiments of the invention. However, the invention is not limited to the precise arrangements and instrumentalities of the embodiments depicted in the drawings. Further, as provided, like reference numerals contained in the drawings are meant to identify similar or identical elements.

FIG. 1 is an exploded view illustration showing the components comprising one possible embodiment of a limited use ultrasonic coupling device of the present disclosure. This particular embodiment resembles a bandage with a mechanical break feature to render the device unusable following treatment.

FIG. 2A is a rendering showing the embodiment of FIG. 1 in one configuration. Foil seals on the top and bottom of a coupling medium chamber preserve the coupling medium for proper ultrasound transmission during storage and shipping.

FIG. 2B is a rendering showing the embodiment of FIG. 1 with foil seals removed. In this state, the coupling device may accept an ultrasonic transducer and may be mechanically coupled to the body for therapy.

FIG. 2C is a rendering showing the embodiment of FIG. 1 with foil seals removed and ultrasonic transducer fastened to the coupling device. The coupling device may then be received onto a body for ultrasonic treatment.

FIG. 3A is an illustration depicting one embodiment of a coupling bandage. This bandage may have any shape, but is shown here with four symmetric lobes. This particular shape is beneficial for symmetric or rounded locations on a body.

FIG. 3B is an illustration depicting a “Y” shaped coupling bandage used mainly in non-symmetric body locations and joints.

FIG. 3C is an illustration depicting a modified “Y” shaped coupling bandage which may be used on joints with the bottom portion of the “Y” positioned on the articulating surfaces of the joint.

FIG. 4A is an illustration showing an ultrasonic transducer mechanically fastened to an inner ring of the ultrasonic coupling device. The ultrasonic coupling device exhibits a break tab and a perforated line at the base of the coupling device for rendering the coupling device unusable following the end of a treatment.

FIG. 4B is an illustration showing an ultrasonic transducer mechanically fastened to an outer ring of the ultrasonic coupling device. A simple rounded snap-fit is utilized to provide easy insertion and removal of the ultrasonic transducer. This may be modified to provide a hooked snap-fit for difficult removal of the ultrasonic transducer without damaging the coupling device.

FIG. 5 is an illustration of a gel puck. The gel puck may be mechanically coupled to the ultrasonic transducer and the ultrasonic transducer mechanically coupled to a wrap, brace, bandage, or other fixture. The gel puck may also be mechanically coupled directly to the wrap, brace, bandage, or other fixture. A disposable mechanism can be applied to the gel puck and or fixture.

FIG. 6A is a rendering of one embodiment of the present invention. An ultrasonic transducer is mechanically coupled, via snap fit, to a coupling device which has a thinned or perforated area. The mechanical fit is such that prevents removal of the ultrasonic transducer without damaging the coupling device. Following a treatment, the ultrasonic transducer may be twisted to break the thinned or perforated area, which releases the ultrasonic transducer from the coupling device and renders the coupling device unusable.

FIG. 6B is a rendering of the cross-section of the embodiment described in FIG. 6A. The cross-section shows the one-way mechanical snapping feature of the coupling device. The cross-section further shows the perforated area and a slotted area in the ultrasonic transducer that interacts with a boss on the coupling device to deliver a shear force on the perforated area.

FIG. 6C is a rendering of another cross-section of the ultrasonic coupling device embodiment described in FIG. 6A. This cross-section shows the interaction of the ultrasonic transducer with the coupling device in the slotted regions. This interaction creates the shear force needed to break the perforated area of the coupling device.

FIG. 7 shows a series of renderings of one embodiment of the ultrasonic coupling device. This embodiment utilizes four areas for mechanically fastening an ultrasonic transducer to the coupling device. These four areas are surrounded by thinned or mechanically weak areas. When the ultrasonic transducer is removed from the coupling device, the upward pull force is converted into a transverse force which breaks the thinned areas surrounding the mechanical fasteners. This allows removal of the ultrasonic transducer and renders the ultrasonic coupling device unusable by preventing subsequent ultrasonic transducer connections.

FIG. 8 shows a rendering of one embodiment of the present invention. This embodiment resembles that of FIG. 6; however the perforated or thinned area is at the intersection of the coupling medium chamber and the patient coupling area. When the perforation is broken, the coupling chamber can no longer be held to the patient and the coupling device is rendered unusable.

FIG. 9 is a series of renderings of one embodiment of the present invention that utilizes an electromechanical method. An ultrasonic transducer is fastened onto the coupling device, in this case, with a screw threading. Upon turning the ultrasonic transducer completely onto the coupling device, an internal boss of the ultrasonic transducer causes an angled boss of the coupling device to engage an electromechanical switch that allows ultrasonic treatment to begin.

FIG. 10 is a drawing of one embodiment of the present invention that utilizes an electromechanical means of activating and limiting the use of the ultrasonic treatment. A tactile switch is mounted on the ultrasonic transducer. The ultrasonic transducer is then mechanically fastened to the coupling device. The tactile switch is activated by a rigid or semi-rigid coupling medium that allows activation of the ultrasonic treatment. Software controls can ensure that the device is removed following treatment completion.

FIG. 11 is a drawing of one embodiment of the present invention that utilizes an electromechanical means of activating and limiting the use of the ultrasonic treatment. A tactile switch is mounted on the ultrasound ultrasonic transducer. When the ultrasonic transducer is mechanically fastened to the coupling device, the tactile switch is depressed by the support wall of the ultrasound coupling medium chamber, which activates the ultrasonic treatment.

FIG. 12 is a series of renderings of one embodiment of the present invention that utilizes an electromechanical means of activating and limiting the use of the ultrasonic treatment. A membrane switch is placed between the PCB and ultrasound transducer within the ultrasound ultrasonic transducer. The ultrasonic transducer is depressed slightly when mechanically fastened to the coupling device, which depresses the internal membrane switch and activates the ultrasonic treatment.

FIG. 13 is a series of renderings of one embodiment of the present invention that utilizes an electromechanical means of activating and limiting the use of the ultrasonic treatment. Once the ultrasonic transducer is mechanically fastened to the coupling device, a secondary connector is inserted, which completes a circuit and activates the ultrasonic treatment.

FIG. 14 is an illustration depicting a coupling device with embedded electrically conductive ring. An ultrasonic transducer with two external conducting terminals contact the coupling device conductive ring when fully coupled together. This completes a circuit and activates the ultrasonic treatment.

FIG. 15 is an illustration depicting one embodiment of the present invention that utilizes an electromechanical means of activating and limiting the use of the ultrasonic treatment. A tactile switch is mounted inside the ultrasound ultrasonic transducer. When the ultrasonic transducer is mechanically fastened to the coupling device, the tactile switch is depressed by the area of the ultrasound coupling device used for coupling to a patient's body. Depressing the switch allows activation of the ultrasonic treatment.

FIG. 16 is an illustration that depicts a passive or active electrical component implanted within the fabric of the coupling device. The electrical component(s) comprise a means of wireless communication (e.g. RFID, NFC, BLE) from the coupling device to the ultrasonic transducer.

FIG. 17 is an illustration that depicts a passive or active electrical component implanted within the ultrasonic coupling medium chamber. The electrical component(s) comprise a means of wireless communication (e.g. RFID, NFC, BLE) from the coupling device to the ultrasonic transducer.

FIG. 18 is an illustration of a coupling device that contains an ultrasonic coupling medium with a narrow impedance spectrum. An impedance analyzer within the ultrasonic transducer is brought into contact when the ultrasonic transducer is coupled to the coupling device. The impedance of the coupling medium may be continuously or periodically monitored to ensure proper acoustic coupling and safety.

### DETAILED DESCRIPTION OF THE INVENTION

The present invention relates to an ultrasound coupling adapter, an ultrasound coupling device comprising the ultrasound coupling adapter, and an ultrasound coupling system comprising the ultrasound coupling device and an ultrasound transducer, as further described herein. The present invention also relates to various ultrasound kits and ultrasound coupling systems configured to include the ultrasound coupling adapter and device of the present invention. Further, the present invention relates to various methods of using and making the ultrasound coupling adapter, device, and system of the present invention.

The ultrasound coupling adapters, devices, and systems of the present invention have various attributes, as described more fully herein. In particular embodiments, the ultrasound coupling adapter, device, and system are configured for limited use or one-time use. Without meaning to limit the present invention to a particular embodiment, provided below are various attributes of the present invention.

In one aspect, the present invention provides an ultrasound coupling adapter for coupling an ultrasound transducer to an ultrasound coupling medium. As used herein, the term “ultrasound coupling adapter” may also be referred to as an “ultrasound coupling compartment” or other terms that one of ordinary skill in the art would understand as referring to the “ultrasound coupling adapter” as more fully described below. The ultrasound coupling adapter comprises: an interface support region for operably interfacing the ultrasound transducer to the ultrasound coupling medium; and an integrated means for (i) rendering the ultrasound coupling adapter inoperable, and/or (ii) preventing operation of the ultrasound transducer when not properly coupled with the ultrasound coupling adapter and/or ultrasound coupling medium.

As used herein, the term “interface support region” refers to any structure that is effective for operably interfacing an ultrasound transducer to an ultrasound coupling medium. In certain embodiments, the interface support region can be configured as a single component. However, in other embodiments, the interface support region can be made of multiple components that, when combined, are effective for operably interfacing an ultrasound transducer to an ultrasound coupling medium.

Without intending to limit the scope of the invention, in certain embodiments, the interface support region has a dual purpose of providing a housing region for the ultrasound coupling medium, and providing a structural support region for contacting with and/or positioning the ultrasound transducer for operable interfacing with the ultrasound coupling medium. In such embodiments, the “housing region” functions to locate, orient, position, and/or partially or fully constrain the ultrasound coupling medium for purposes of interfacing with the ultrasound transducer. Examples of suitable interface support regions are illustrated in various drawings of the present disclosure, as further described below.

As used herein, the term “housing region” may also be referred to as an “inner chamber” or “compartment” or similar such term. In such embodiments, a term such as “inner chamber” is meant to describe an area within the inner boundaries of the interface support region, with the interface support region providing a solid structure or boundary for housing the ultrasound coupling medium.

In another aspect, the present invention provides an ultrasound coupling device that comprises: an ultrasound coupling adapter according to the present disclosure; and an ultrasound coupling medium housed in the ultrasound coupling adapter. The integrated means is for rendering the ultrasound coupling adapter inoperable, thereby rendering the ultrasound coupling device inoperable. In certain embodiments, the ultrasound coupling device further comprises an adhesive fabric for interfacing the ultrasound coupling device with a subject, where the fabric has adhesive properties that substantially diminish after first use by the subject. In certain other embodiments, the ultrasound coupling device further comprises an adhesive fabric for interfacing the ultrasound coupling device with a subject, where the fabric has adhesive properties that diminish by 50% or greater using the ASTM D903 standard.

In one embodiment of the ultrasound coupling device of the present disclsosure, the integrated means functions to permanently alter the interface support region or the ultrasound coupling medium. In certain embodiments, the integrated means is configured so that permanently altering the interface support region is achieved by bending, twisting, tearing, pulling, or pushing a feature of the integrated means.

In one embodiment, the integrated means is selected from the group consisting of a pull tab, a perforated or thinned area, a snap-fit tab, and a stress concentrator, said integrated means being configured to facilitate permanently altering a portion of the interface support region.

In one embodiment, the pull tab serves as a lever capable of rendering the ultrasound coupling device inoperable.

In one embodiment, the perforated or thinned area completely or partially extends around the perimeter of the outer support region of the ultrasound coupling adapter.

In one embodiment, the interface support region comprises a key and keyseat pair configuration with an ultrasound transducer that interface together to facilitate the permanent altering of the configuration.

In one embodiment, the interface support region comprises a threaded portion that couples with an ultrasound transducer, where the threaded portion prevents decoupling with the ultrasound transducer unless at least a portion of said interface support region is permanently altered.

In one embodiment, the perforated or thinned areas are adjacently disposed to the snap-fit tab that enable permanent deformation of the configuration upon insertion or removal of an ultrasound transducer.

In one embodiment, the integrated means comprises thermal, chemical, or ultrasound-assisted degradation. In certain embodiments, the degradation is controlled by the thickness and chemical composition of the interface support region or ultrasound coupling medium.

In another aspect, the present invention provides an ultrasound coupling system comprising: an ultrasound coupling device according to the present disclosure; and an ultrasound transducer configured for operable attachment to the ultrasound coupling device.

In another aspect, the present invention provides a method of regulating application of ultrasound energy to a subject, where the method comprises the steps of: applying ultrasound energy to a subject using an ultrasound coupling system of the present disclosure; and manipulating the integrated means of the ultrasound coupling adapter so as to render the ultrasound coupling device inoperable, thereby causing the ultrasound energy to cease being applied to the subject. In one embodiment of this method, the manipulating step is performed by the subject or triggered by the integrated means itself

In another aspect, the present invention provides an ultrasound coupling device comprising: an ultrasound coupling adapter according to the present disclosure; and an ultrasound coupling medium housed in the ultrasound coupling adapter, where the integrated means is for preventing operation of an ultrasound transducer when not properly coupled with the ultrasound coupling adapter and/or ultrasound coupling medium.

The integrated means can comprise an electromechanical, electrical, or optical means.

In one embodiment, the electromechanical means comprises a switch selected from the group consisting of a slide, toggle, rotary, rocker, knife, pushbutton, and membrane. According to certain embodiments, the switch is located on or within the ultrasound transducer and is positioned to interact with the ultrasound coupling adapter, the ultrasound coupling medium, the subject, or the ultrasound transducer, or with an enclosure or lens in which the ultrasound transducer is affixed.

In one embodiment of the ultrasound coupling device, the electrical means functions by completing a circuit by connecting contacts located on an ultrasound transducer using a metallic or other electrically conductive material.

The electrically conductive material can be constructed to at least partially extend around the perimeter of the interface support region of the ultrasound coupling adapter.

In one embodiment of the ultrasound coupling device, the optical means functions by interrupting or completing an optical switch when the ultrasound transducer is mechanically connected to the ultrasound coupling device.

In another aspect, the present invention provides an ultrasound coupling system comprising: an ultrasound coupling device according to the present disclosure; and an ultrasound transducer configured for operable attachment to the ultrasound coupling device.

In another aspect, the present invention provides a method of regulating application of ultrasound energy to a subject, where the method comprises the steps of: (i) operably coupling the ultrasound transducer to the ultrasound coupling adapter and/or ultrasound coupling medium, such that said operably coupling manipulates the integrated means, whereby the manipulation of the integrated means allows activation of the ultrasound coupling system; and (ii) applying ultrasound energy to a subject using the activated ultrasound coupling system. In one embodiment of this method, the manipulating step is performed by the subject or triggered by the integrated means itself

In another aspect, the present invention provides an ultrasound coupling device comprising: an ultrasound coupling adapter according to the present disclosure; and an ultrasound coupling medium housed in the ultrasound coupling adapter, where the integrated means is for both (i) rendering the ultrasound coupling adapter inoperable and (ii) preventing operation of the ultrasound transducer when not properly coupled with the ultrasound coupling adapter and/or ultrasound coupling medium.

The integrated means of this ultrasound coupling device can comprise an electromechanical, electrical, or optical means.

In one embodiment, the electrical means comprises either passive or active components selected from the group consisting of a radio frequency identification (RFID) tag, a near field communication (NFC) tag, a Bluetooth module, a Bluetooth low energy (BLE) module, a Wireless Fidelity (Wi-Fi) module, a ZigBee module, a cellular module, or other wireless technologies, with supporting electronics selected from the group consisting of batteries, memory, microcontrollers, field programmable gate arrays (FPGA), and programmable logic devices (PLD).

In one embodiment, the electrical means includes a passive or active radio frequency identification (RFID) or Near Field Communication (NFC) tag that contain either a unique or generalized access code that is received by a reader located in an ultrasound system containing the device.

In one embodiment, the tag access code is erased following commencement or termination of an ultrasound treatment of the subject.

In one embodiment, the tag is embedded within or adhered to the interface support region of the ultrasound coupling adapter, the ultrasonic coupling medium, or an adhesive fabric in close proximity to the tag reader when an ultrasound transducer is mechanically connected to the ultrasound coupling device.

In one embodiment, the electrical means includes an ultrasound transducer that directly or indirectly determines the electrical impedance of the ultrasound coupling medium through electrical contacts.

In one embodiment, the device functions with an ultrasound system that includes a barcode, Quick Response (QR) code, or similar reader, an infrared sensor, or an optical sensor.

In one embodiment, the optical means functions to scan a unique access code by an ultrasound system in the form of a barcode or Quick Response (QR) code located on the ultrasound coupling device, permit system operation, and restrict system operation after a predetermined period of time.

In one embodiment, the optical means comprises an optical sensor that measures the color or opacity of the ultrasound coupling medium.

In one embodiment, the ultrasound coupling medium is compounded with an oxygen or ultrasound-sensitive molecule that changes the color or opacity of the ultrasound coupling medium.

In one embodiment, the optical means comprises an infrared sensor that measures the rate of change and absolute value of temperature of the ultrasound coupling medium.

In one embodiment, the optical means comprises an optical sensor that measures the index of refraction or back scatter of the ultrasound coupling medium.

In another aspect, the present invention provides an ultrasound coupling system comprising: an ultrasound coupling device according to the aspect of the present disclosure as described above; and an ultrasound transducer configured for operable attachment to the ultrasound coupling device.

In another aspect, the present invention provides a method of regulating application of ultrasound energy to a subject, the method comprising the steps of: applying ultrasound energy to a subject using an ultrasound coupling system according to the aspect of the present disclosure as described above; and manipulating the integrated means of the outer support region of the ultrasound coupling device so as to (i) render the ultrasound coupling device inoperable and (ii) prevent operation of an ultrasound transducer when not properly coupled with the ultrasound coupling adapter and ultrasound coupling medium. In one embodiment, the manipulating step is performed by the subject or triggered by the integrated means itself

In another aspect, the present invention provides an ultrasound coupling device comprising: an ultrasound coupling adapter according to the present disclosure; and an ultrasound coupling medium housed in the ultrasound coupling adapter, where the ultrasound coupling device in a first state is operably connected to an ultrasound transducer and comprises an integrated means of rendering the ultrasound coupling device inoperable, and where the ultrasound coupling device in a second state is inoperable.

In another aspect, the present invention provides a method of regulating application of ultrasound energy to a subject, the method comprising the steps of: applying ultrasound energy to a subject using an ultrasound coupling system comprising an ultrasound transducer coupled to the ultrasound coupling device according to the aspect of the present disclosure as described above, where ultrasound energy is applied when the ultrasound coupling device is in the first state, and where ultrasound energy is not applied when the ultrasound coupling device is in the second state.

In one embodiment of this method, first and second states are achieved by an action performed by the subject or triggered by the ultrasound coupling device.

The drawings referred to herein are for the purposes of illustrating the various aspects of the present invention and are not meant to limit the scope of the present invention. Below is a description of aspects and embodiments of the present invention as illustrated in the accompanying drawings.

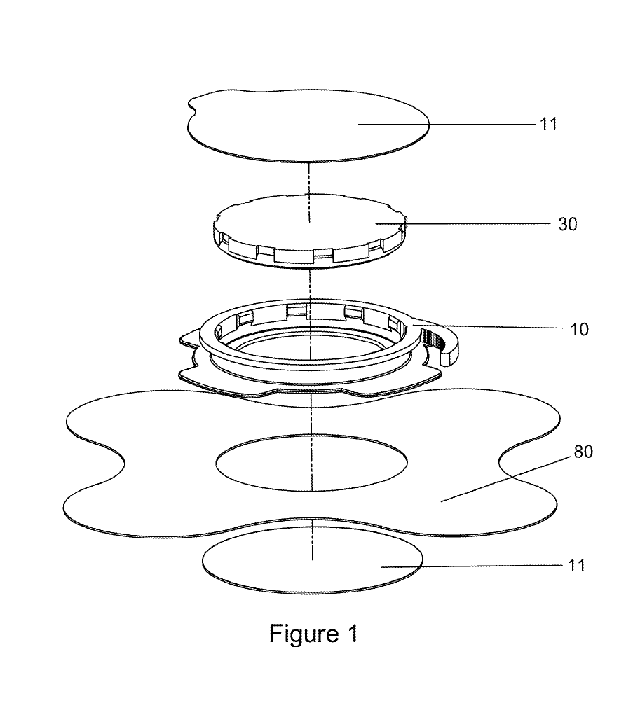

Referring now to FIG. 1 in particular, as well as to various aspects of FIGS. 1-18, there is shown an exploded view illustration of one embodiment of an ultrasound coupling device 50 comprising a top foil seal 11, an ultrasound coupling medium 30, an ultrasound coupling adapter 10, an adhesive fabric 80, and a bottom foil seal 11. The top and bottom foil seals 11 seal to the ultrasound coupling adapter 10 to isolate the ultrasound coupling medium 30 with a water-tight barrier from the atmosphere. The foil seal 11 may be composed of, but not limited to, aluminum, tin, stainless steel, or copper. The foil seal 11 may be co-laminated with various other materials that may be composed of, but not limited to, polyethylene, polypropylene, wax, polyethylene terephthalate, polyimide, acrylic, or silicone. These co-laminates allow bonding to the ultrasound coupling adapter 10 based on the material composition of the ultrasound coupling adapter 10. The bonding to the ultrasound coupling adapter 10 and/or to the adhesive fabric 80 may be accomplished through induction or conduction heat seal, ultrasound welding, friction welding, or laser beam welding. The adhesive fabric 80 may be composed of, but not limited to, a non-woven polymer fabric, woven fabric, elastomeric fabric, polyester fabric, polypropylene fabric, rayon, nylon, or other synthetic materials, or polyurethane foam.

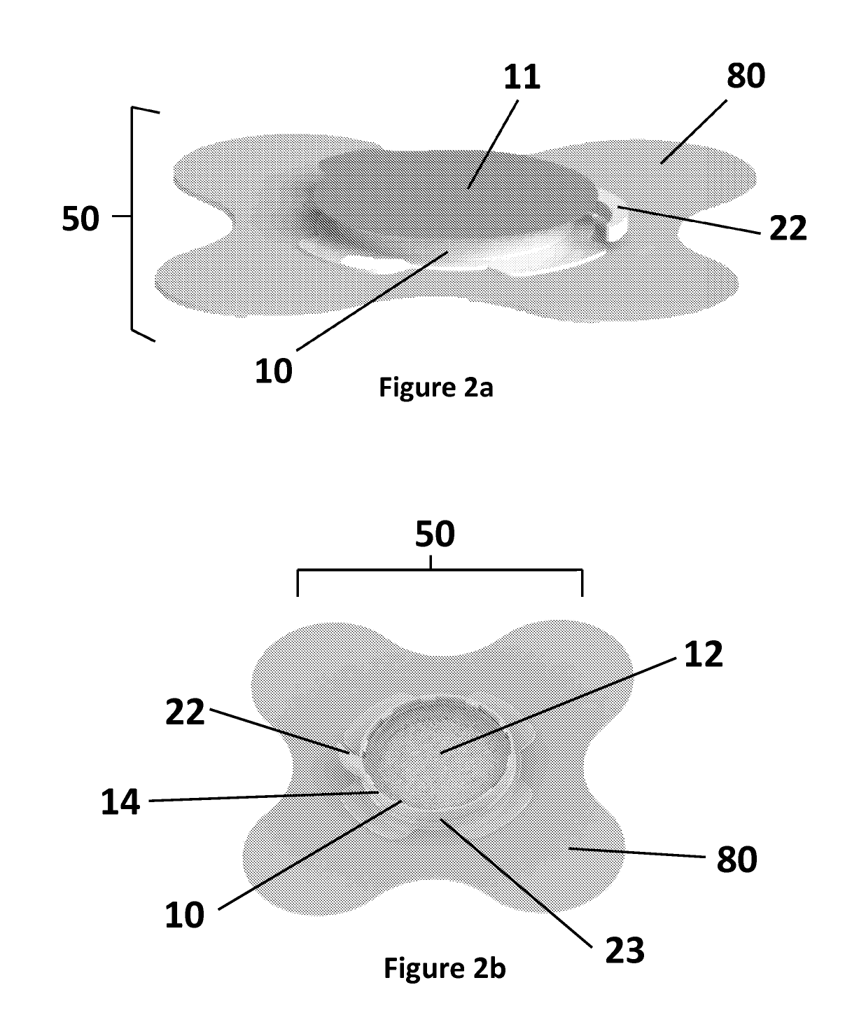

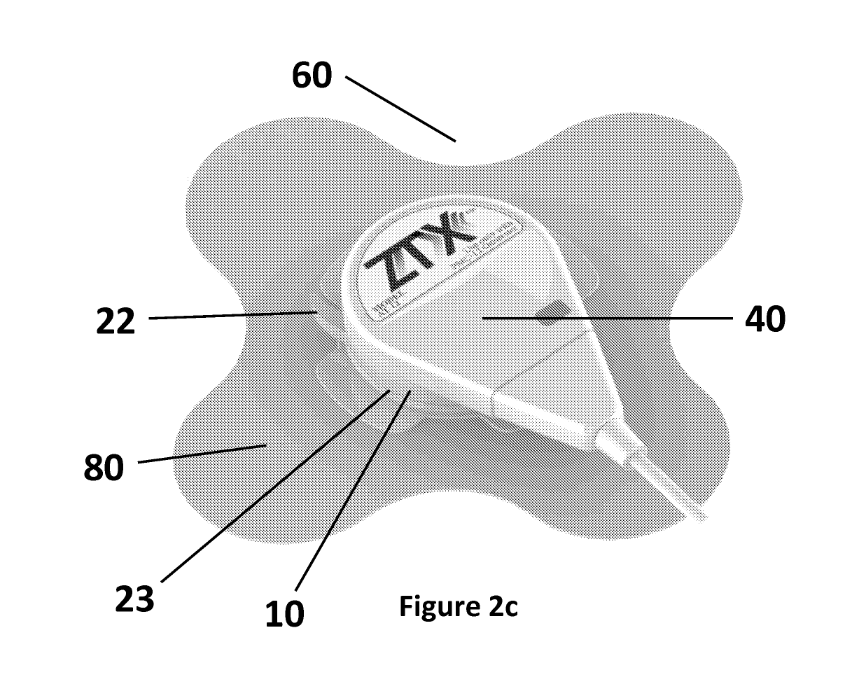

The ultrasound coupling medium 30 may be comprised of water, ultrasound gel, or hydrogel. The ultrasound coupling medium 30 is designed to transmit ultrasound effectively at frequencies ranging from 20 kHz to 40 MHz and may be cured ex situ, in situ, or may not require curing. The ultrasound coupling medium 30 is designed such that it contacts the ultrasound transducer 40 (see, e.g., FIG. 2C) and surface of a patient's body in the area requiring treatment, imaging, or diagnosis. The coupling of an ultrasound transducer 40 to the ultrasound coupling device 50 (see, e.g., FIGS. 2A-2C) can protrude the ultrasound coupling medium 30, which allows coupling to concave portions of a patient's body.

As shown in FIGS. 1-4, the ultrasound coupling adapter 10 serves multiple functions, it: mechanically connects the ultrasound coupling device 50 to the ultrasound transducer 40 by way of an interface support region 14, maintains the shape of the ultrasound coupling medium 30, provides sealing surfaces for the foil seals 11, and may or may not provide a means for dislodging the ultrasound transducer 40 that renders the ultrasound coupling device 50 inoperable following treatment termination. In one embodiment, the interface support region 14 comprises snap fit tabs 24 on the internal ring of the ultrasound coupling adapter 10. The snap fit tabs 24 may be designed for two-way or one-way operation, that is, removable or permanent connection to the ultrasound transducer 40, respectively. With a one-way connection of the ultrasound coupling adapter 10 to the ultrasound transducer 40, the ultrasound coupling adapter 10 includes a pull tab 22. The pull tab 22 may be any shape or size with pull direction tangential, radial, horizontal, or vertical, and provides leverage for a patient to break the ultrasound coupling adapter 10. In one embodiment, the pull tab 22 is curved with a thinned or perforated area 23 underneath the pull tab 22. The thinned or perforated area 23 may continue partially or completely around the circumference or perimeter of the ultrasound coupling adapter 10. Upon applying force to the pull tab 22, a shear force is generated in the thinned or perforated area 23 causing the material to separate and ultrasound coupling adapter 10 to dislodge the ultrasound transducer 40. The breaking of the ultrasound coupling adapter 10 prevents future ultrasound transducers 40 from mounting to the ultrasound coupling adapter 10 and thus prevents old or dried ultrasound coupling medium 30 from being improperly used in treatment.

In another embodiment, two perforated or thinned areas 23 may be located above and below the pull tab 22. When applying force to the pull tab 22, a shear force is generated in the thinned or perforated areas 23 and causes both areas to split. In this embodiment, the whole structure is not destroyed, but just a portion of the ultrasound coupling adapter 10. The portion of the ultrasound coupling adapter 10 destroyed removes the mechanical feature of the interface support region 14, in this case a snap-fit tab 24, that fastens the ultrasound transducer 40 to the ultrasound coupling adapter 10. The removal of the mechanical feature of the interface support region 14 dislodges the ultrasound transducer 40 from the coupling device.

The ultrasound coupling adapter 10 cross-sectional shape may be selected from, but not limited to, the group comprising: a circle, square, oval, hexagon, octagon, rectangle, triangle, or any other shape to match or accept an ultrasound transducer 40 of various shapes and sizes. The ultrasound coupling adapter 10 material composition may be comprised of, but not limited to, polyethylene, polypropylene, ABS, polycarbonate, silicone, or santoprene. The ultrasound coupling adapter 10 could be made from a stiff material for treatment in flat areas. The ultrasound coupling adapter 10 may also be made from a flexible or pliable material for conformance to curved or concave areas of treatment. The ultrasound coupling adapter 10 may also be designed with reliefs or unique cross-sections that allow compliance and are independent of material selection.

FIGS. 2A-2C show renderings of the embodiment from FIG. 1. FIG. 2A represents one embodiment of the ultrasound coupling device 50 during storage and/or shipping. The foil seals 11 seal the ultrasound coupling medium 30 in a water-tight chamber. When the therapy is being prepared, the foil seals 11 are removed, exposing the ultrasound coupling medium 30 as in FIG. 2B. FIG. 2C shows an ultrasound transducer 40 mechanically connected to the ultrasound coupling device 50. The ultrasound coupling device 50 can then be positioned on a patient's body in a location requiring treatment. Alternatively, the ultrasound coupling device 50 of FIG. 2B may be positioned on a patient's body in a location requiring treatment followed by connection of the ultrasound transducer 40 to the ultrasound coupling device 50.

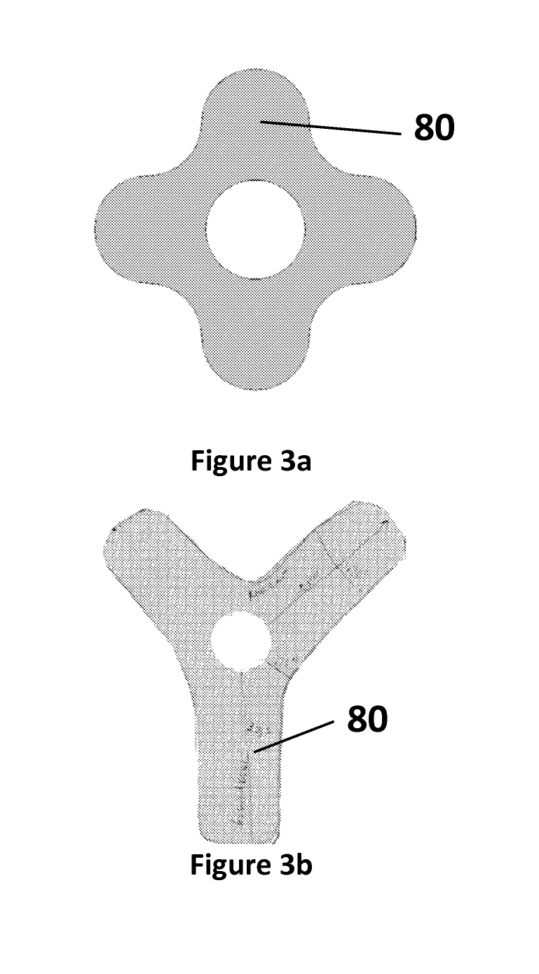

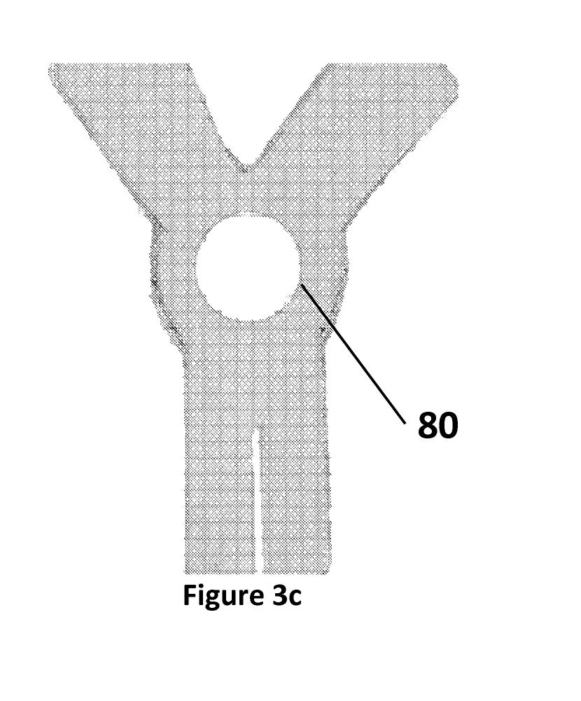

FIGS. 3A-3C show illustrations and drawings depicted various shapes of the ultrasound coupling device 50 adhesive fabric 80 as disclosed in FIG. 1. FIG. 3A depicts a four-lobed symmetric adhesive fabric 80. The lobes create a larger surface area for adherence of the ultrasound coupling device 50 to the patient's body. The symmetry of the shape is typically used on flat or larger areas of the body, but may be used everywhere. FIG. 3B depicts a “Y” shaped adhesive fabric 80. This shape is typically used in asymmetric surfaces and on joints. The tail of the “Y” is placed on the articulating surfaces of the joint. FIG. 3C depicts a modified “Y” shaped adhesive fabric 80. The bottom or tail portion of the “Y” is split into two strips and is placed on the articulating surfaces of the joint. The “Y” shaped adhesive fabrics 80 provide relief when moving the joints and prevent pulling and discomfort while treating the joints.

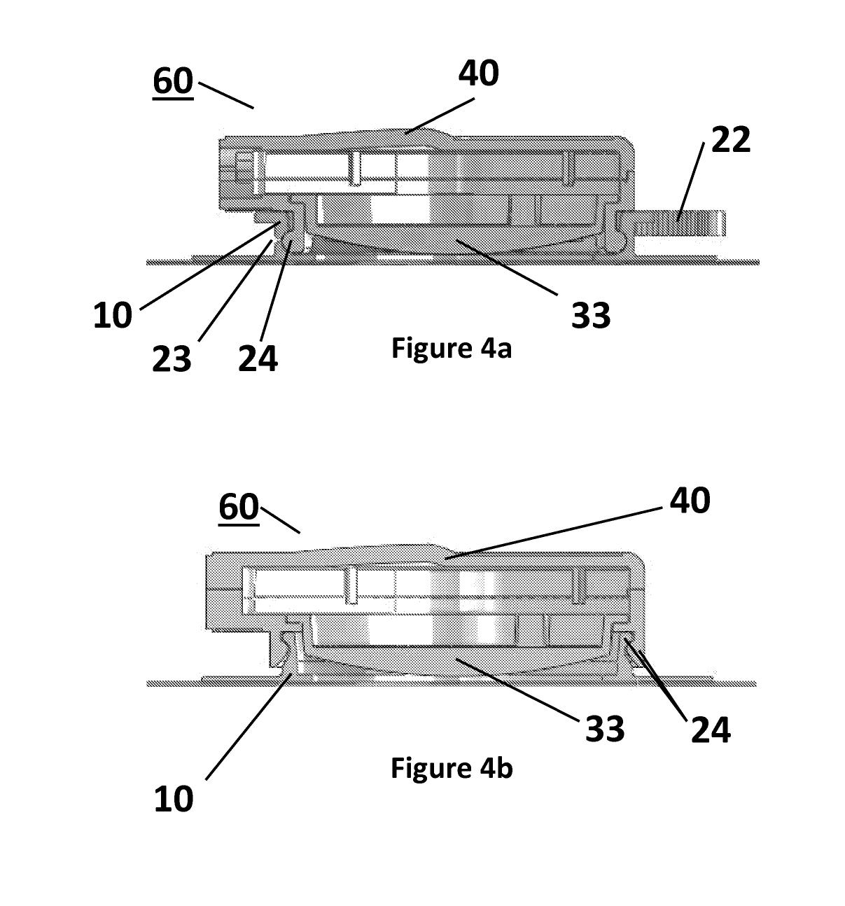

FIGS. 4A-4B show renderings of two embodiments of the ultrasound coupling adapter 10 with two methods for mechanically connecting the coupling bandage to an ultrasound transducer 40 by way of the interface support region 14. FIG. 4A depicts a mechanical snap-fit tab 24 connection on the inner portion of the ultrasound coupling adapter 10. The ultrasound transducer 40 is then connected internally within the inner chamber 12. FIG. 4B depicts a mechanical snap-fit tab 24 connection on the outer portion of the ultrasound coupling adapter 10. The ultrasound treatment is then connected externally to the inner chamber 12. FIG. 4A and 4B depict a two-way mechanical connection where the ultrasound transducer 40 may be connected and disconnected. However, another embodiment may comprise hooked or angled snap-fit tab 24 projections which serve as a one-way mechanical connection.

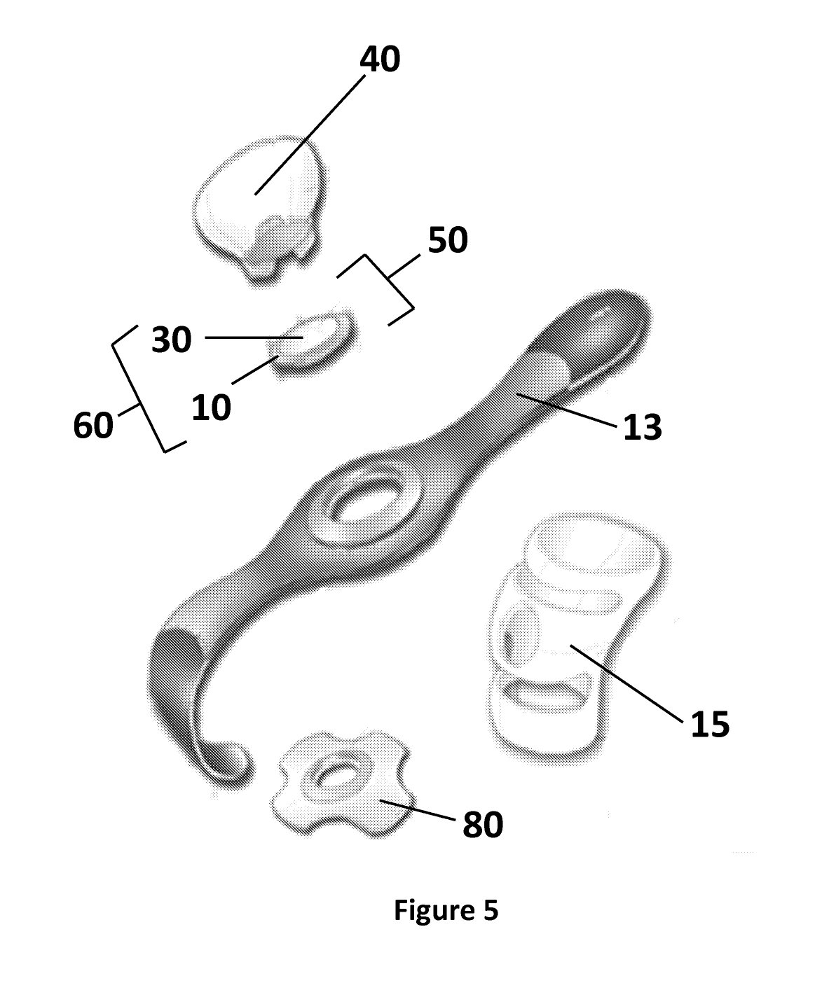

FIG. 5 is an illustration of the ultrasound coupling device 50 that may be mechanically coupled to an ultrasound transducer 40. In turn, the ultrasound transducer 40 is mechanically coupled to a wrap 13, adhesive fabric 80, brace 15, or other fixturing device. Alternatively, the ultrasound coupling device 50 may mechanically couple to both the ultrasound transducer 40 and either a wrap 13, adhesive fabric 80, brace 15, or other fixturing device by way of the interface support region 14. One embodiment of the ultrasound coupling device 50 comprises a top foil seal 11, ultrasound coupling adapter 10, ultrasound coupling medium 30, and bottom foil seal 11, similar to that disclosed in FIG. 1. The ultrasound coupling adapter 10 houses, and provides structure to, the ultrasound coupling medium 30. The foils seals 11 seal the inner chamber 12 and create a water-tight barrier to the atmosphere during storage and shipping of the ultrasound coupling device 50.

Another embodiment of the ultrasound coupling device 50 comprises a ultrasound coupling adapter 10 and ultrasound coupling medium 30. The ultrasound coupling adapter 10 provides mechanical stability and may have mechanisms to prevent repeated use of the ultrasound coupling device 50, such as pull tab 22 or other mechanism disclosed in this invention. Evaporation of water in different ultrasound coupling media 30 would cause shriveling of the media and prevent further use of the ultrasound coupling device 50 in treatment.

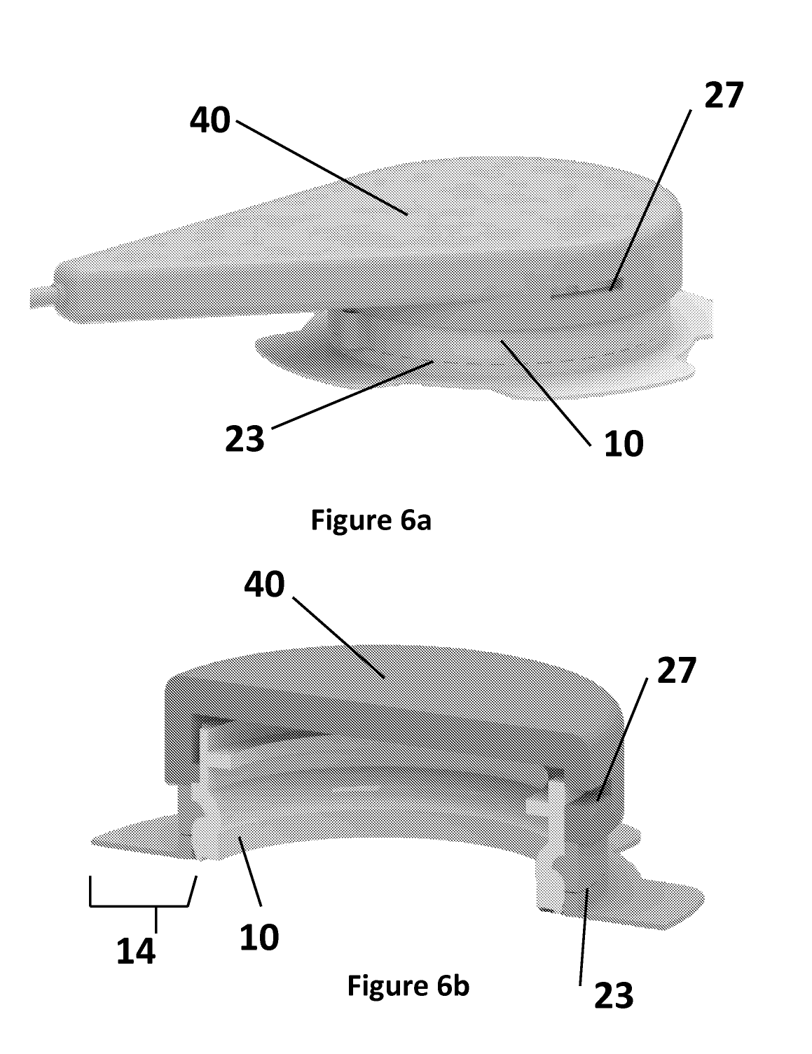

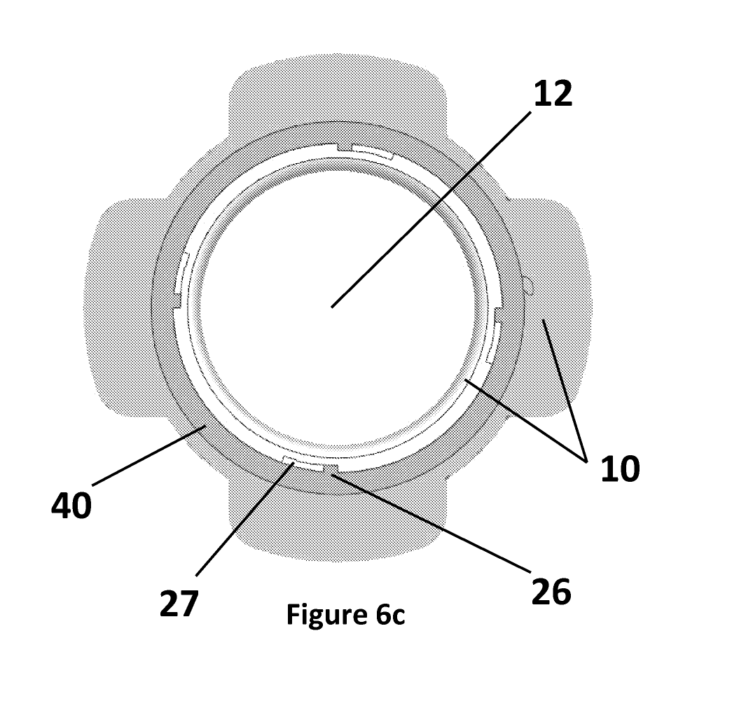

FIGS. 6A-6C are renderings of one embodiment of the limited use ultrasound coupling adapter 10 coupled to an ultrasound transducer 40 that uses a mechanical means for rendering the ultrasound coupling system 60 inoperable. FIG. 6A depicts an ultrasound transducer 40 mechanically connected to one embodiment of the ultrasound coupling adapter 10. A perforated of thinned area 23 is shown at ⅓ the height of the ultrasound coupling adapter 10. The perforated or thinned area 23 may appear anywhere on the height of the ultrasound coupling adapter 10. Furthermore, the perforated or thinned area 22 may extend completely or partially around the circumference of the ultrasound coupling adapter 10. In embodiments where the ultrasound coupling adapter 10 is not circular, the perforated or thinned area 22 may extend completely or partially around the perimeter of the ultrasound coupling adapter 10. In another embodiment, the perforated or thinned area 22 may not be confined to a single plane and may curve upward or downward to complete the destruction of the ultrasound coupling adapter 10.

FIG. 6B is a cross-section of the ultrasound coupling adapter 10 connected to the ultrasound transducer 40. A one-directional mechanical snap-fit tab 24 is shown as the mechanical method for connecting the ultrasound coupling adapter 10 to the ultrasound transducer 40. In this case, a method of removing the ultrasound transducer 40 by destroying the ultrasound coupling adapter 10 is required. In another embodiment, the mechanical snap-fit tab 24 may be two-directional, allowing for the ultrasound transducer 40 to be removed.

FIG. 6C is a cross-section of the ultrasound coupling adapter 10 parallel to the top surface of the ultrasound coupling adapter 10. The cross-section is taken at the leverage point for breaking the ultrasound coupling adapter 10. A slotted portion, otherwise known as a keyseat 27, on the external surface of the ultrasound coupling adapter 10 fits a boss, otherwise known as a key 26, from the ultrasound transducer 40. Once the ultrasound transducer 40, or ultrasound coupling adapter 10, is rotated in relation to the other, the key 26of the ultrasound transducer 40 is pressed against the end of the keyseat 27 in the ultrasound coupling adapter 10, which creates a shear force in the perforated or thinned area 23. The shear force allows for the breaking of the ultrasound coupling adapter 10, rendering the ultrasound coupling adapter 10, and thus the ultrasound coupling device 50 inoperable. The breaking of the ultrasound coupling adapter 10 further permits removal of the ultrasound transducer 40 for subsequent use with new ultrasound coupling adapters 10. In another embodiment, the ultrasound transducer 40 may have the keyseat 27 while the ultrasound coupling adapter 10 comprises the key 26 for fitting into the keyseat 27 and creating the leverage point for breaking the ultrasound coupling adapter 10. Furthermore, the leverage point, i.e. the key 26 and keyseat 27 pair, may be anywhere on the height of the ultrasound coupling adapter 10 or even on the base of the ultrasound coupling adapter 10 (i.e. opposite the patient contacting surface).

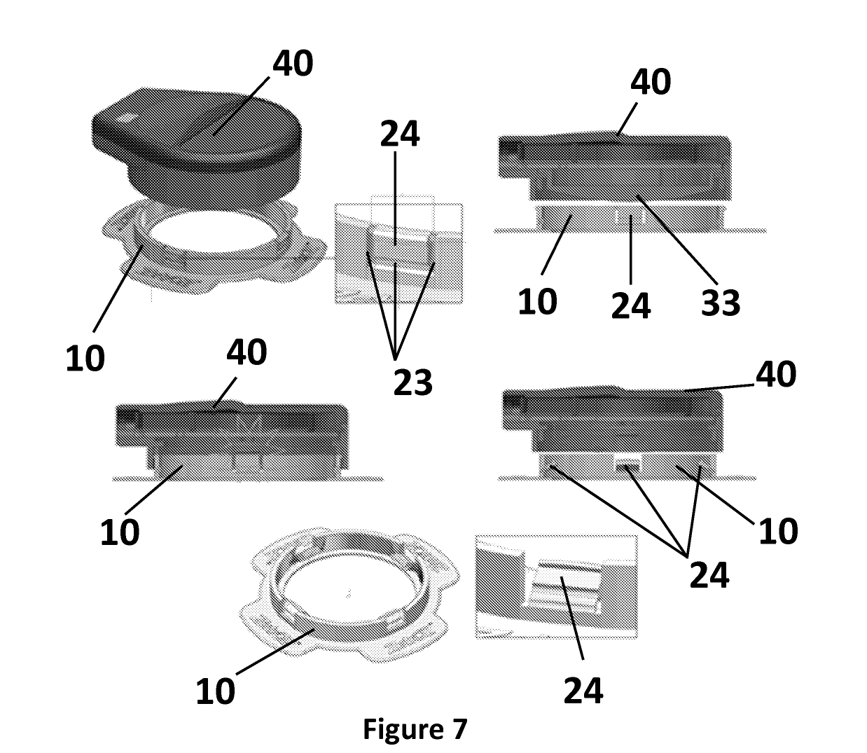

FIG. 7 is one embodiment of the present invention utilizing a mechanical means to render the ultrasound coupling adapter 10 inoperable. The ultrasound coupling adapter 10 comprises an interface support region 14that consists of four snap-fit tabs 24. The snap-fit tab 24 feature allows connection to the ultrasound transducer 40. These four snap-fit tabs 24 are surrounded by thinned or perforated areas 23. When the ultrasound transducer 40 is removed, the upward pull of the ultrasound transducer 40 imparts a transverse force onto the snap-fit tabs 24. The transverse force is large enough to break the thinned or perforated areas 23 surrounding the snap-fit tabs 24. This breaks the interface support region 14, and thus the mechanical connection, freeing the ultrasound transducer 40, and rendering any further uses of the ultrasound coupling adapter 10 futile. The number of snap-fit tabs 24 may increase or decrease in quantity or size depending on the strength of connection required and decoupling force desired.

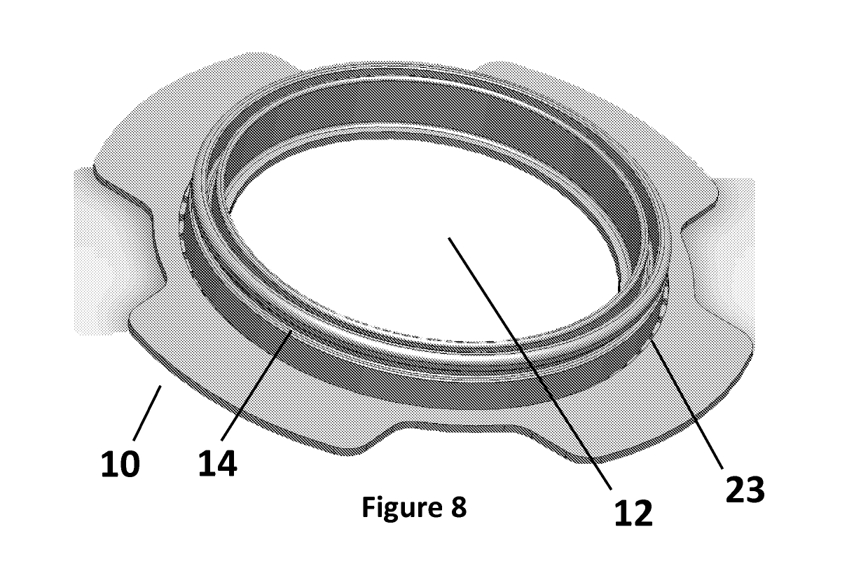

FIG. 8 is one embodiment of the present invention utilizing a perforated or thinned area 23 that is located on the base of the ultrasound coupling adapter 10 (i.e. the portion that contacts the patient's body). The thinned or perforated area 23 may be present at any location on the base and may extend completely or partially around the perimeter of the ultrasound coupling adapter 10. In one embodiment, bosses on the ultrasound transducer 40 and ultrasound coupling adapter 10 interface support region 14 intertwine with each other, like cogs in a gear, and create a leverage point for breaking the thinned or perforated area 23 when the ultrasound transducer 40 or ultrasound coupling adapter 10 is rotated in relation to the other.

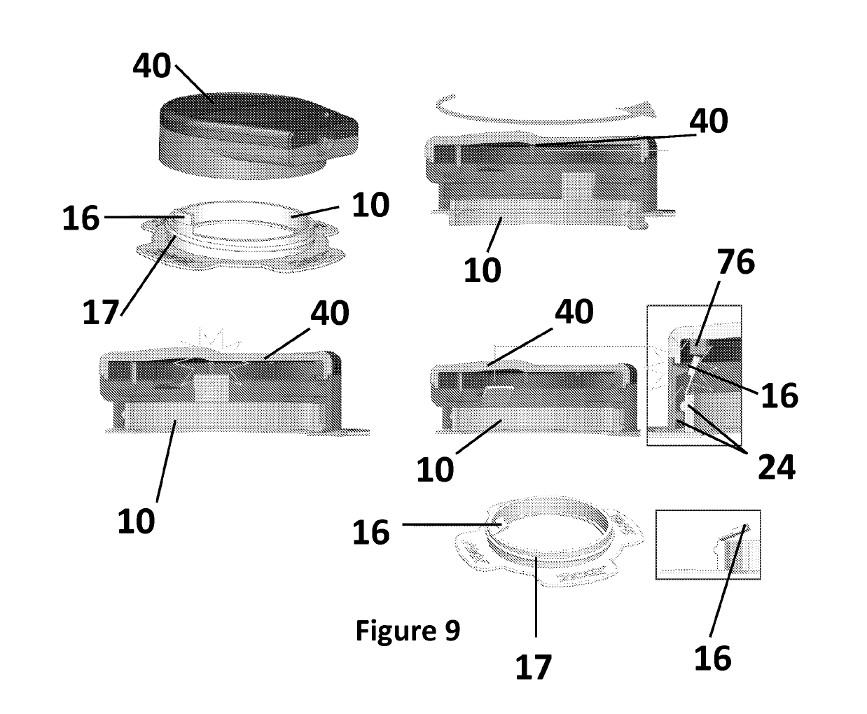

FIG. 9 is one embodiment of the present invention that incorporates both mechanical and electrical safety features. The ultrasound coupling adapter 10 consists of a threaded region 17 and vertical flange 16. An ultrasound transducer 40 is screwed onto the ultrasound coupling adapter 10 to bring the ultrasound transducer 40 in contact with the ultrasound coupling medium 30. When the ultrasound transducer 40 is completely screwed into place, a boss on the ultrasound transducer 40 dislodges the vertical flange 16 from the ultrasound coupling adapter 10, which angles the flange 16 and brings it into contact with a tactile switch 76 located within the ultrasound transducer 40. The depression of the tactile switch 76 either completes or breaks an internal circuit located on a printed circuit board (PCB) 18 within the ultrasound transducer 40 signaling that treatment may be administered safely. When the ultrasound transducer 40 is removed, the flange 16 on the ultrasound coupling adapter 10 is permanently deformed, inhibiting any further use of the ultrasound coupling adapter 10 in subsequent treatments. The activation switch 76 may further be selected from the group of switches comprising slide, toggle, rotary, rocker, knife, pushbutton, membrane, optical, infrared, or the like.

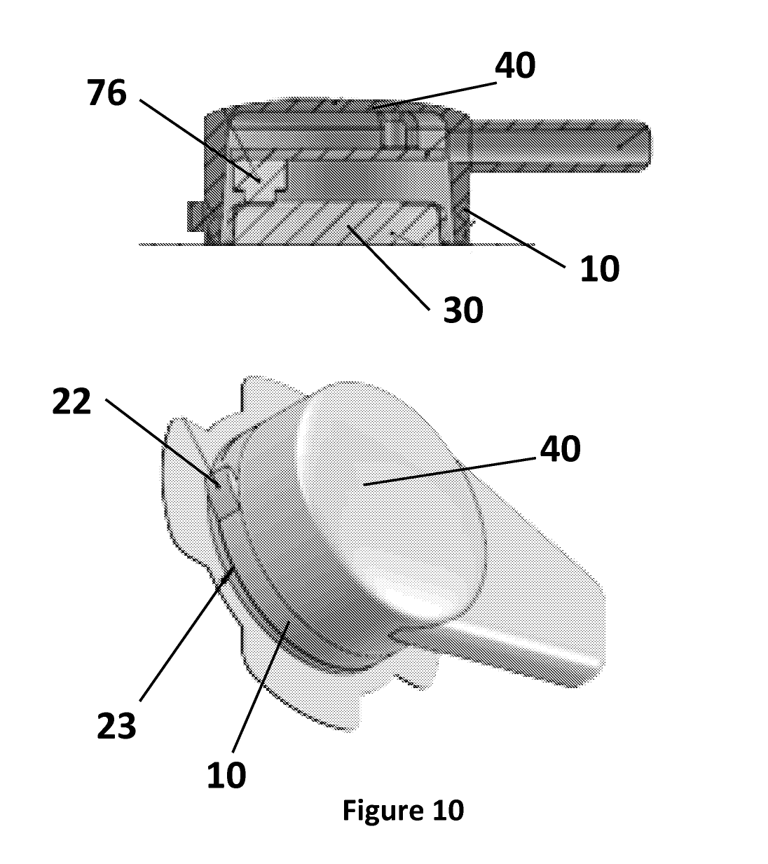

FIG. 10 is one embodiment of the ultrasound ultrasound coupling system 60 that includes a switch 76 embedded in the ultrasound transducer 40. The switch is located above the ultrasound coupling medium 30 of the ultrasound coupling device 50. When the ultrasound transducer 40 is mechanically connected to the ultrasound coupling adapter 10, as disclosed in this invention, the switch 76 is depressed by the ultrasound coupling medium 30. The ultrasound coupling medium 30 is comprised of a hydrogel or other semi-solid or solid material that allows efficient transmission of ultrasound. The stiffness of the ultrasound coupling medium 30 allows for the depression of the switch 76 located in the ultrasound transducer 40. Once the switch 76 is depressed, a PCB 18 circuit is either connected or broken, allowing for the activation of a treatment. As the ultrasound coupling medium 30 dries and shrinks, the contact to the switch 76 is lost and further treatment cannot be activated.

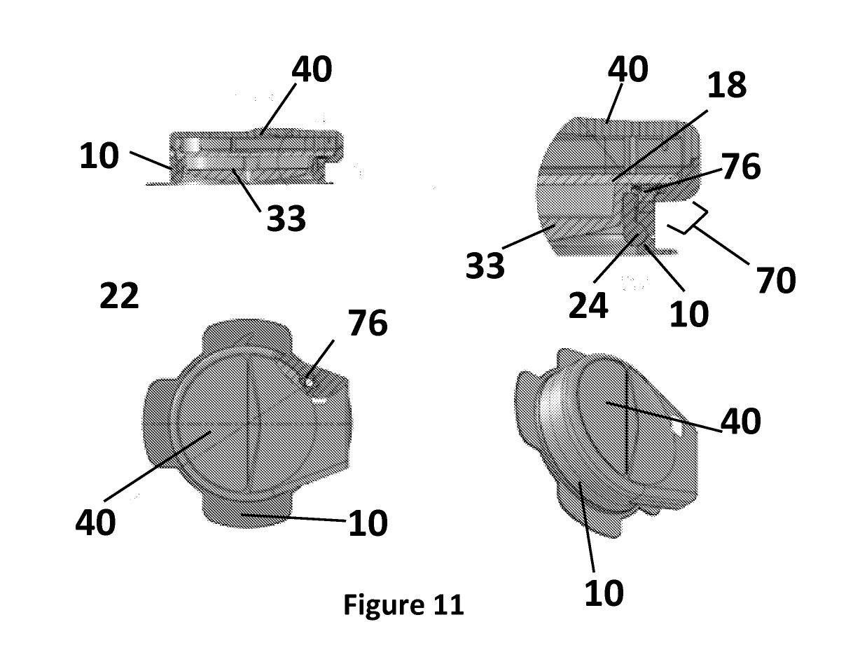

In another embodiment of the present invention, FIG. 11 depicts an electromechanical means 70 to activate an ultrasound transducer 40 using a switch 76 that is embedded in the ultrasound transducer 40 and is situated above the ultrasound coupling adapter 10 sidewall. When the ultrasound transducer 40 is mechanically connected to the ultrasound coupling adapter 10 by way of the interface support region 14, the ultrasound coupling adapter 10 depresses the switch 76 on the ultrasound transducer 40 allowing for activation of an ultrasound treatment. The switch 76 may be present at any location in the ultrasound transducer 40 that is situated above the perimeter of the ultrasound coupling adapter 10.

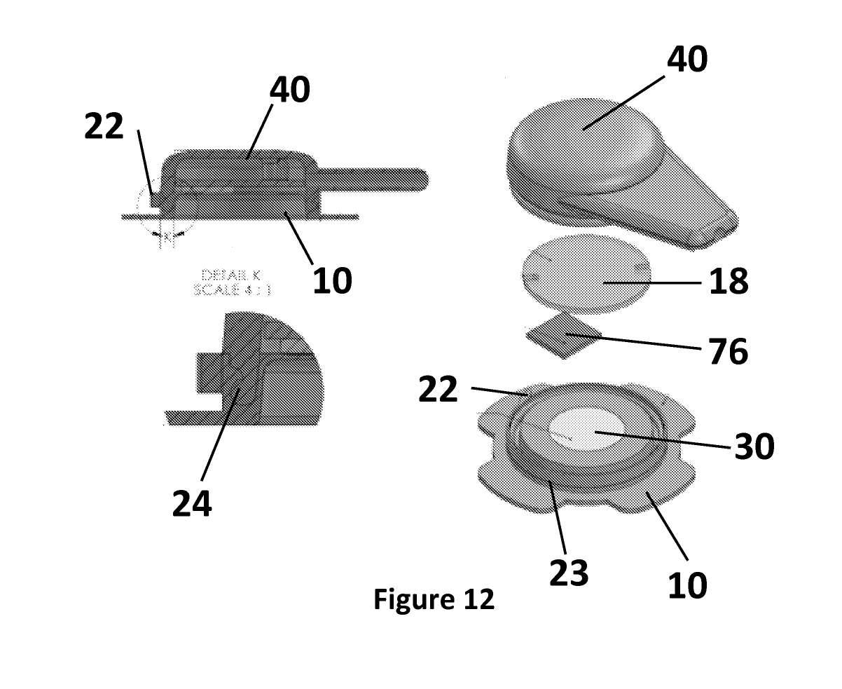

In another embodiment of the present invention, FIG. 12 depicts a switch 76 located between the PCB 18 and the ultrasound transducer 40. The ultrasound transducer 40, or the lens 33 or enclosure in which the ultrasound transducer 40 is affixed, is allowed to translate more proximal or more distal to the mounted switch 76. The ultrasound coupling device 50 comprises the ultrasound coupling adapter 10, for mechanically connecting the ultrasound transducer 40, and an ultrasound coupling medium 30. The ultrasound coupling medium 30 may be stiff or slightly compressible, such as with hydrogels, or other solid or semi-solid materials that are transmissive to ultrasound. The stiffness of the ultrasound coupling medium 30 imparts force on the ultrasound transducer 40, or the lens 33 or enclosure in which the ultrasound transducer 40 is affixed, causing translation toward the mounted switch 76 in the ultrasound transducer 40. The translation of the ultrasound transducer 40 depresses the switch 76 and allows activation of an ultrasound treatment. Alternatively, the ultrasound coupling adapter 10 of the ultrasound coupling device 50 may have a mechanical boss that imparts a force on the lens 33, enclosure, or ultrasound transducer 40 of the and similarly cause depression of the switch 76. With a mechanical boss that translates the lens 33, enclosure, or ultrasound transducer 40 of the, ultrasound coupling media 30 of any viscosity or stiffness may be used. Furthermore, upon removal of the ultrasound coupling adapter 10 from the ultrasound transducer 40, the mechanical boss may be permanently deformed, such that reconnection of the ultrasound transducer 40 with the same ultrasound coupling adapter 10 does not activate treatment.

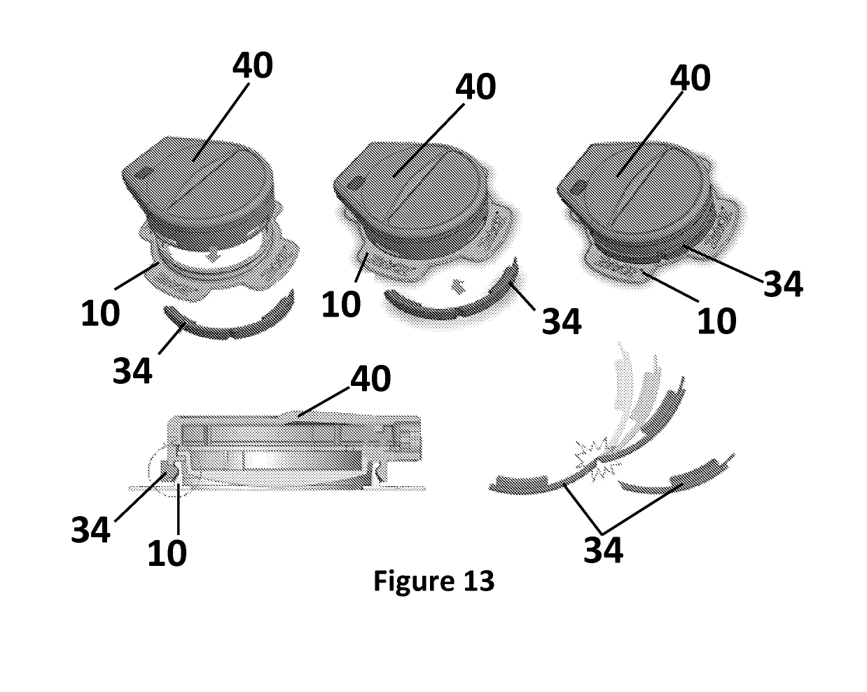

FIG. 13 depicts one embodiment of the present invention that includes an ultrasound transducer 40, ultrasound coupling adapter 10, and a third connecting component 34. The ultrasound transducer 40 is mechanically connected to the ultrasound coupling adapter 10 using methods disclosed in the current invention. A third connecting component 34 is inserted into the assembly that further connects the ultrasound transducer 40 and ultrasound coupling adapter 10. The connecting component 34 also acts as a means to activate the ultrasound treatment. In one embodiment, the connecting component 34 depresses a mechanical switch 76 on the ultrasound transducer 40 once inserted. Depression of the switch 76 either breaks or connects an internal circuit that allows activation of the treatment. In another embodiment, the connecting component 34 may be metallic or electrically conductive to electrically connect two or more contacts 21 located on the ultrasound transducer 40. In yet another embodiment, the connecting component 34 may be a flange with living hinge that is integral with the ultrasound coupling adapter 10 of the. The ultrasound coupling adapter 10 limits the use of the ultrasound transducer 40 by requiring that the connecting component 34 be removed to begin another treatment. Upon removal, the connecting component 34, or the ultrasound coupling adapter 10, is damaged and inhibits further treatment using the same ultrasound coupling adapter 10.

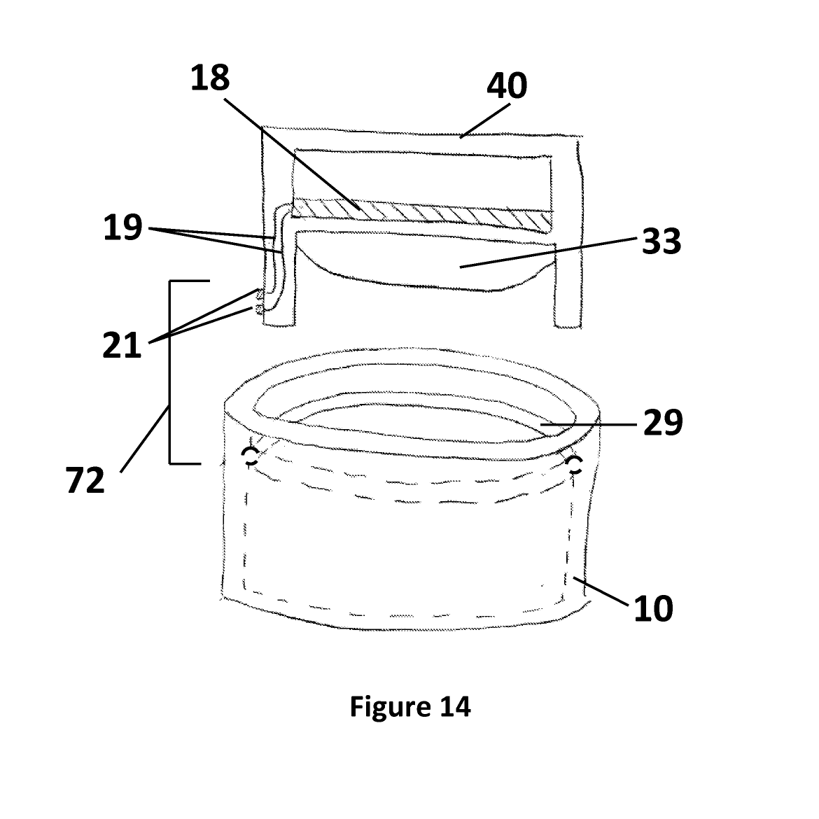

FIG. 14 shows an ultrasound coupling adapter 10 with embedded metallic or electrically conductive ring 29. The conductive ring 29 may extend completely or partially around the perimeter of the ultrasound coupling adapter 10. The conductive ring 29 may further be located at any dimension along the height of the ultrasound coupling adapter 10. When the ultrasound transducer 40 is connected to the ultrasound coupling adapter 10, electrical means 72 are used to either activate the treatment, in the form of two or more contacts 21 on the ultrasound transducer 40 electrically connected by the conductive ring 29 on the ultrasound coupling adapter 10. The contacts 21 are electrically connected to a PCB 18 with or without the use of wires 19. The connection of the two or more contacts 21 on the ultrasound transducer 40 completes a circuit internal to the ultrasound transducer 40 that allows activation of the ultrasound treatment. In another embodiment, the electrically conductive ring 29 may be any shape that completely or partially extends around the perimeter of a ultrasound coupling adapter 10.

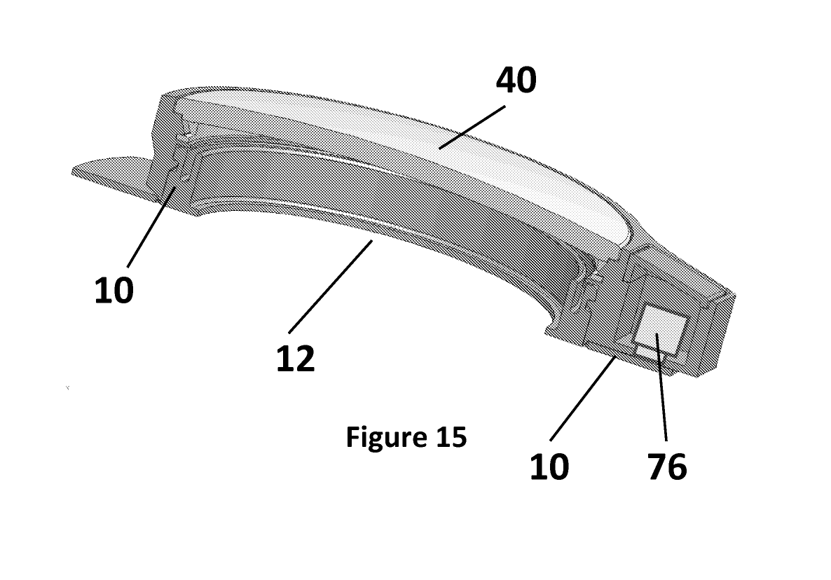

FIG. 15 depicts one embodiment of the present invention where a switch 76 is mounted inside the ultrasound transducer 40. The location of the switch 76 is such that when the ultrasound transducer 40 is mechanically connected to the ultrasound coupling adapter 10, the switch 76 is depressed by the base of the ultrasound coupling adapter 10 (i.e. by the portion of the ultrasound coupling adapter 10 extending radially out from the chamber sidewall). In the embodiment shown in FIG. 15, the switch 76 contacts the surface of the ultrasound coupling adapter 10 base. When the switch 76 is depressed, an internal circuit to the ultrasound transducer 40 is either broken or completed, which allows activation of the ultrasound treatment. In another embodiment, the switch 76 may be depressed by an adhesive fabric 80 or by the patient surface.

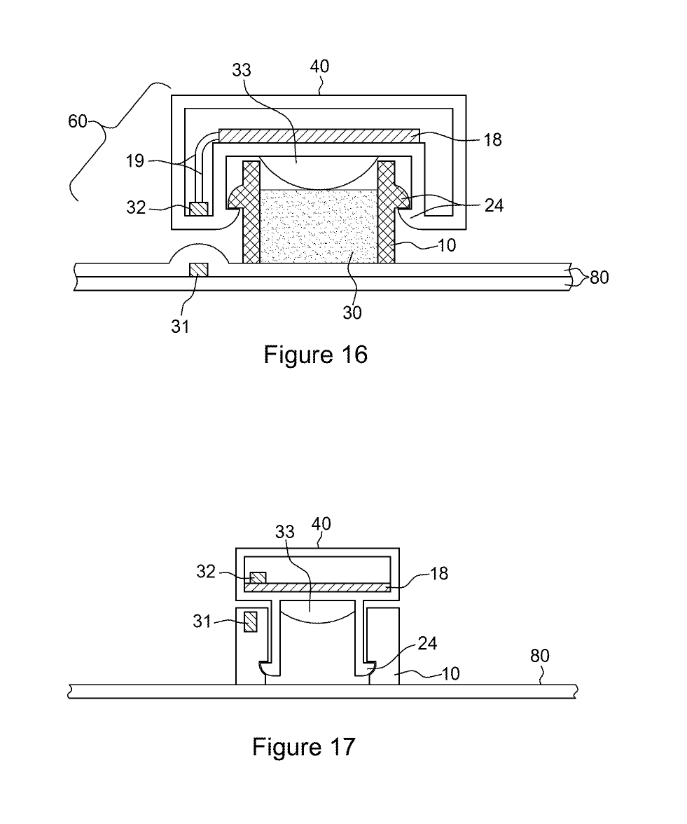

FIG. 16 depicts the use of wireless methods to communicate, and activate, an ultrasound transducer 40. One embodiment of the ultrasound coupling adapter 10 comprises an ultrasound coupling adapter 10 with or without foil seals 11, ultrasound coupling medium 30, and a adhesive fabric 80. The adhesive fabric 80 is composed of four layers of material. The first and second layers are a non-woven or similar fabric. The third layer is an adhesive layer that permits attachment to a patient's body. The adhesive may comprise acrylics, silicones, or the like. The fourth layer is a paper liner that prevents premature adhesion to other materials. In between the first and second layer, at least one electrical component is deposited. The electrical component may consist of a Near Field Communication (NFC) tag 31, Radio Frequency Identification (RFID) tag, Bluetooth module, Bluetooth Low Energy (BLE) module, Wireless Fidelity (Wi-Fi) module, Zigbee module, cellular module, cloud module, or other wireless communication modules, a power source, memory, or the like. The adhesive fabric 80 may have more or less than four layers and the at least one electrical component may be deposited at any location in between any two layers or on top or bottom of the adhesive fabric 80.

In one embodiment, an NFC tag 31 is used within the adhesive fabric 80 and is positioned in such a way that it is in close proximity to the coupled ultrasound transducer 40. The NFC tag 31 may be active, requiring a power source, or passive, only requiring the tag itself The NFC tag 31 may contain a predetermined access code that, once read by the NFC reader 32 technology embedded in the ultrasound transducer 40, will verify the access code and permit activation of the ultrasound treatment. The access code may be similar across all ultrasound coupling devices 50, or may have a unique access code for each ultrasound coupling device 50. With unique access codes, the NFC reader 32 will ensure that no duplicate ultrasound coupling devices 50 may be used past the predetermined life of the ultrasound coupling device 50. Unique access codes may further be transmitted and checked against a cloud database of codes. With a generalized access code for all ultrasound coupling devices 50, an NFC write operation from the NFC reader 32 located in the ultrasound transducer 40 can erase the access code in the NFC tag 31 following a successful read of the NFC tag 31 and activation of the ultrasound treatment. With the NFC tag 31 erased, the ultrasound coupling device 50 could not be used in further treatments. The power source used for active tags and devices may consist of lithium polymer, lithium ion, nickel cadmium, or other common battery types. The power source may further consist of devices that harvest electrostatic potentials on a patient's body or devices that use movement or temperature of a patient's body to develop a potential to power electrical components.

In another embodiment, a Bluetooth low energy (BLE) module deposited on the ultrasound coupling adapter 10 may communicate with the ultrasound transducer 40 to activate the ultrasound treatment. Additionally, a main control module of the ultrasound transducer 40 may be used as the activation point for ultrasound treatment.

In another embodiment of the present invention, FIG. 17 depicts an NFC tag 31, RFID tag, or other wireless communication modules embedded within the inner chamber 12 or within the ultrasound coupling adapter 10. An NFC tag 31 may be overmolded with a plastic such that the ultrasound coupling adapter 10 is formed through injection molding with an embedded tag. Besides the location and manufacturing method for positioning a tag within the inner chamber 12 or ultrasound coupling adapter 10, the basic mechanism of use is similar to FIG. 16.

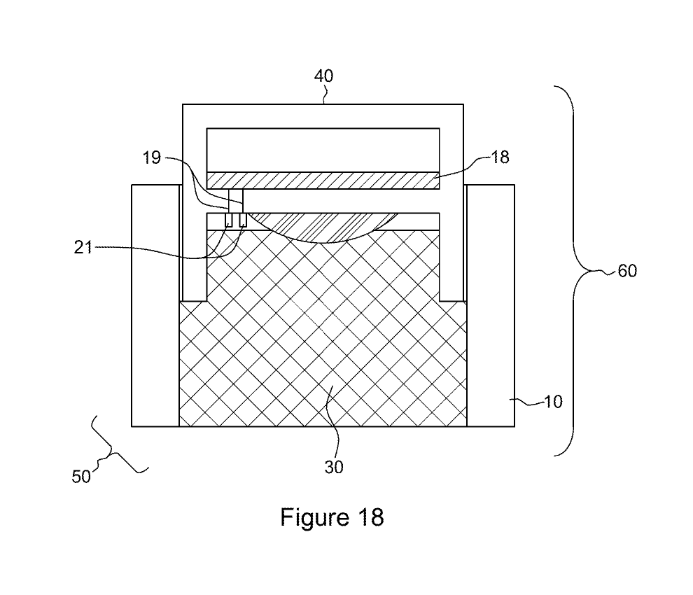

FIG. 18 depicts an electrical means 72 of analyzing the ultrasound coupling medium 30 of the ultrasound coupling device 50 to determine if further treatment is safe. The ultrasound transducer 40 consists of two electrical contacts 21 that interact with the ultrasound coupling medium 30. A series of low current sinusoidal waves are swept through a number of frequencies related to the ultrasound frequency delivered. By sweeping the frequencies and analyzing the voltage and/or current differences, the electrical impedance of the ultrasound coupling medium 30 can be discerned. Based on preset values, the ultrasound transducer 40 would prevent activation of a treatment if the electrical impedance is below the preset value. Alternatively, the swept frequencies may be unrelated to the frequency of ultrasound used in the treatment.

In another embodiment, the sinusoidal wave used to drive the ultrasound transducer 40 is swept through different frequencies. Depending on the voltage and current values measured during the different frequencies, the electrical impedance of the ultrasound transducer 40, and thus the acoustic impedance of the system, can be determined and compared against preset values. If the electrical/acoustic impedance is determined to be lower than the preset value, further treatments would be prevented using the current ultrasound coupling device 50.

One embodiment of the present invention comprises a ultrasound coupling adapter 10 with at least one embedded electrical component. The at least one electrical component consists of other safety and functionality mechanisms that would limit the furtherance of treatment using the current ultrasound coupling adapter 10. In one embodiment, a thermocouple or temperature sensor is embedded into the ultrasound coupling adapter 10. The rate of heating, maximum temperature, and time above a predetermined temperature may be tracked within the ultrasound coupling adapter 10 or the data transmitted to the ultrasound transducer 40 or ultrasound control module. In ultrasound coupling devices 50 with less effective transmission of ultrasound energy, more heat would be dissipated and would be measured by the temperature sensor embedded within the ultrasound coupling adapter 10. The ultrasound transducer 40 would compare the temperature measurements to preset values and would limit further treatment using the current ultrasound coupling adapter 10.

One embodiment of the present invention involves a chemical means to limit the use of the ultrasound coupling device 50. The ultrasound coupling device 50 comprises an ultrasound coupling adapter 10, ultrasound coupling medium 30, and the option of foil seals 11. The ultrasound coupling medium 30 chemical composition is such that degradation of the ultrasound coupling medium 30 occurs after a prescribed time of use. The degradation may be activated or assisted through increased heat, ultrasound energy, a chemical compound, or a combination of the aforementioned. In one embodiment, the ultrasound coupling medium 30 is composed of two or more layers that are individually transmissive to acoustic energy. Upon activating the ultrasound treatment, mixing of the chemicals occurs, through ultrasound-assisted acoustic streaming, causing the ultrasound coupling medium 30 to shrink in volume and the ultrasound coupling device 50 to become inoperable. In another embodiment, the ultrasound coupling medium 30 volume remains the same, but the molecular structure changes causing inefficient transmission of acoustic energy, which warrants changing the ultrasound coupling device 50. The change in molecular structure may be signaled by a color or opacity change that signals the user to change the ultrasound coupling device 50.

In yet another embodiment, the ultrasound coupling adapter 10 comprises a material, such as PLA, PLGA, or the like, that degrades through chemical, thermal, or ultrasound-assisted means. The design and material composition of the ultrasound coupling adapter 10 may be designed such that the degradation time is predetermined. When the ultrasound coupling adapter 10 degrades, the integrity of the interface support region 14 is compromised and the mechanical connection to an ultrasound transducer 40 is prevented.

Another embodiment of the present invention is an ultrasound coupling adapter 10 with a magnetic source embedded within the adhesive fabric 80. When the ultrasound transducer 40, or control module, is connected to, or brought in close proximity to, the ultrasound coupling adapter 10, respectively, a circuit senses the magnetic energy and allows activation of the ultrasound treatment. In another embodiment, the magnetic source may be placed within the inner chamber 12 or embedded within the ultrasound coupling adapter 10.

In another embodiment, an adhesive fabric 80 may be used to couple the ultrasound coupling adapter 10 to a location on the patient's body. After one or a predefined number of treatments, the adhesive properties of the adhesive fabric 80 substantially diminish to prevent further coupling of the ultrasound coupling adapter 10 to the patient. In one embodiment, the adhesive properties of the adhesive fabric 80 are diminished by 40% or greater after one use according to the ASTM D903 standard.

One embodiment of the present invention is a method of using an optical means of limiting the use of the ultrasound coupling device 50. The ultrasound coupling device 50 consists of an ultrasound coupling adapter 10, ultrasound coupling medium 30, and an adhesive fabric 80. A barcode or Quick Response (QR) code may be embedded on the adhesive fabric 80 or ultrasound coupling adapter 10. When the ultrasound transducer 40 is connected to the ultrasound coupling adapter 10, a barcode of QR code reader visually identifies the code of the ultrasound coupling device 50 and permits activation of the ultrasound treatment if the code is found in a database or is not identical to a previously scanned code. The scanning of the code may also be completed before mechanical connection of the ultrasound transducer 40 to the ultrasound coupling adapter 10. Alternatively, the ultrasound control module may read the code and subsequently permit activation of the ultrasound transducer 40. Additionally, the ultrasound coupling adapter 10 may comprise foil seals 11 that include a barcode or QR code for activation of the ultrasound coupling device 50. After scanning of the code and activation of the treatment, an internal microchip of the ultrasound transducer 40 may limit the use of the ultrasound coupling device 50 to a predetermined period time and prevent further activation of the treatment with the same code.

In another embodiment, an optical switch is located in the ultrasound transducer 40. When the ultrasound transducer 40 is mechanically connected to the ultrasound coupling adapter 10, the optical switch is interrupted. This interruption signals to the ultrasound transducer 40 that treatment activation is safe. Once removed, the ultrasound coupling adapter 10 may permanently deform, which prevents interruption of the optical switch in subsequent attempts to reuse the ultrasound coupling adapter 10.

In another embodiment, an optical switch is located in the ultrasound transducer 40. When the ultrasound transducer 40 is mechanically connected to the ultrasound coupling adapter 10, the optical switch is completed. This may be accomplished by a spring or flange that normally interrupts the optical switch, but is altered upon connection to the ultrasound coupling adapter 10, to allow completion of the optical switch. In another embodiment, the ultrasound coupling adapter 10 may comprise a reflective or angled surface that bends or reflects the light and completes the optical switch. When the ultrasound coupling adapter 10 is removed from the ultrasound transducer 40, the ultrasound coupling adapter 10 is permanently deformed, which prevents the completion of the optical switch in subsequent attempts to reuse the ultrasound coupling adapter 10.

In another embodiment, an optical sensor may be located within the ultrasound transducer 40. The optical sensor may emit light and record the index of refraction of the ultrasound coupling medium 30. Once the ultrasound coupling adapter 10 is connected to the ultrasound transducer 40, the optical sensor is able to measure the index of refraction and compare it to preconfigured limits. As the ultrasound coupling device 50 is used, the moisture content of the ultrasound coupling medium 30 will decrease and the index of refraction will change. When the limit is reached, the ultrasound transducer 40 will not activate until a new ultrasound coupling device 50 is used. In a similar embodiment, the reflection, or back-scattering, of light is measured by the optical sensor. As the ultrasound coupling device 50 is used and the moisture content of the ultrasound coupling medium 30 decreases, the light reflection and/or back-scatter will increase. The measured values are checked against predetermined values and the activation of treatment is prevented when the limit is reached.

In another embodiment, an infrared sensor located in the ultrasound transducer 40 measures the temperature profile of the ultrasound coupling medium 30. The sensor records the maximum temperature, rate of heating, and time spent above a certain temperature to determine the safety and effectiveness of the ultrasound coupling medium 30. Based on predetermined values, the activation of ultrasound is prevented when the limit is reached.

In another embodiment, an optical sensor located in the ultrasound transducer 40 measures the color or opacity of the ultrasound coupling medium 30. As the ultrasound coupling device 50 is used, the moisture content of the ultrasound coupling medium 30 is reduced and the color and/or opacity of the ultrasound coupling medium 30 is changed. This change is measured by the optical sensor and limits the activation of treatment. The color/opacity limit may be a predetermined absolute value or a ratio or percentage of the initial value compared to the final value.

In another embodiment, an oxygen or ultrasound-sensitive chemical or molecule is added to the ultrasound coupling medium 30. The molecule does not interrupt the transmission of ultrasound, but changes the color or opacity of the ultrasound coupling medium 30 after a predetermined exposure to ultrasound and/or oxygen. The change in color or opacity is measured by an optical sensor and the value is compared to a database. When a limit is reached, the ultrasound treatment is prevented until a new ultrasound coupling device 50 is used.

While several aspects of the present invention have been described and depicted herein, alternative aspects may be effected by those skilled in the art to accomplish the same objectives. Accordingly, it is intended by the appended claims to cover all such alternative aspects as fall within the true spirit and scope of the invention.

1. An ultrasound coupling adapter for coupling an ultrasound transducer to an ultrasound coupling medium, said ultrasound coupling adapter comprising:
an interface support region for operably interfacing the ultrasound transducer to the ultrasound coupling medium; and
an integrated means for (i) rendering the ultrasound coupling adapter inoperable, and/or (ii) preventing operation of the ultrasound transducer when not properly coupled with the ultrasound coupling adapter and/or ultrasound coupling medium.

2. An ultrasound coupling device comprising:
an ultrasound coupling adapter according to claim 1; and
an ultrasound coupling medium housed in the ultrasound coupling adapter,
wherein the integrated means is for rendering the ultrasound coupling adapter inoperable, thereby rendering the ultrasound coupling device inoperable.

3. The ultrasound coupling device according to claim 2, wherein the integrated means functions to permanently alter the interface support region or the ultrasound coupling medium.

4. The ultrasound coupling device of claim 3, wherein the integrated means is configured so that permanently altering the interface support region is achieved by bending, twisting, tearing, pulling, or pushing a feature of the integrated means.

5. The ultrasound coupling device according to claim 3, wherein the integrated means is selected from the group consisting of a pull tab, a perforated or thinned area, a snap-fit tab, and a stress concentrator, said integrated means being configured to facilitate permanently altering a portion of the interface support region.

6. The ultrasound coupling device according to claim 5, wherein the pull tab serves as a lever capable of rendering the ultrasound coupling device inoperable.

7. The ultrasound coupling device according to claim 5, wherein the perforated or thinned area completely or partially extends around the perimeter of the outer support region of the ultrasound coupling adapter.

8. The ultrasound coupling device according to claim 3, wherein the interface support region comprises a key and keyseat pair configuration with an ultrasound transducer that interface together to facilitate the permanent altering of the configuration.

9. The ultrasound coupling device according to claim 3, wherein the interface support region comprises a threaded portion that couples with an ultrasound transducer, and
wherein the threaded portion prevents decoupling with the ultrasound transducer unless at least a portion of said interface support region is permanently altered.

10. The ultrasound coupling device according to claim 3, wherein the perforated or thinned areas are adjacently disposed to the snap-fit tab that enable permanent deformation of the configuration upon insertion or removal of an ultrasound transducer.

11. The ultrasound coupling device according to claim 2, wherein the integrated means comprises thermal, chemical, or ultrasound-assisted degradation.

12. The ultrasound coupling device according to claim 11, wherein the degradation is controlled by the thickness and chemical composition of said interface support region or ultrasound coupling medium.

13. The ultrasound coupling device according to claim 2 further comprising:
an adhesive fabric for interfacing the ultrasound coupling device with a subject,
wherein the fabric has adhesive properties that substantially diminish after first use by the subject.

14. The ultrasound coupling device according to claim 2 further comprising:
an adhesive fabric for interfacing the ultrasound coupling device with a subject,
wherein the fabric has adhesive properties that diminish by 50% or greater using the ASTM D903 standard.

15. An ultrasound coupling system comprising:
an ultrasound coupling device according to claim 2; and
an ultrasound transducer configured for operable attachment to the ultrasound coupling device.

16. A method of regulating application of ultrasound energy to a subject, said method comprising the steps of:
applying ultrasound energy to a subject using an ultrasound coupling system according to claim 15; and
manipulating the integrated means of the ultrasound coupling adapter so as to render the ultrasound coupling device inoperable,
thereby causing the ultrasound energy to cease being applied to the subject.

17. (canceled)

18. An ultrasound coupling device comprising:
an ultrasound coupling adapter according to claim 1; and
an ultrasound coupling medium housed in the ultrasound coupling adapter,
wherein the integrated means is for preventing operation of an ultrasound transducer when not properly coupled with the ultrasound coupling adapter and/or ultrasound coupling medium.

19-24. (canceled)

25. An ultrasound coupling system comprising:
an ultrasound coupling device according to claim 18; and
an ultrasound transducer configured for operable attachment to the ultrasound coupling device.

26. A method of regulating application of ultrasound energy to a subject, said method comprising the step of:
operably coupling an ultrasound transducer to an ultrasound coupling adapter and/or ultrasound coupling medium, such that said operably coupling manipulates the integrated means and allows operation of the ultrasound coupling system, whereby ultrasound energy is applied to a subject.

27. (canceled)

28. An ultrasound coupling device comprising:
an ultrasound coupling adapter according to claim 1; and
an ultrasound coupling medium housed in the ultrasound coupling adapter,
wherein the integrated means is for both (i) rendering the ultrasound coupling adapter inoperable and (ii) preventing operation of the ultrasound transducer when not properly coupled with the ultrasound coupling adapter and/or ultrasound coupling medium.

29-46. (canceled)
